(19) United States
(12) Patent Application Publication
(10) Pub. No.: US 2025/0134698 A1
(43) Pub. Date: May 1, 2025

(54) HANDHELD URINE COLLECTION AND PUMP APPARATUS, AND RELATED SYSTEMS AND METHODS

(71) Applicant: PUREWICK CORPORATION, Covington, GA (US)

(72) Inventors: Aaron Van Gorkom, Lynnwood, WA (US); Matthew Jordan Rothberg, Atlanta, GA (US); Claire Gloeckner, Lilburn, GA (US); Marian Gravel, Newburgh, NY (US)

(21) Appl. No.: 18/728,604

(22) PCT Filed: Feb. 1, 2022
(86) PCT No.: PCT/US2022/014749

§ 371 Date: Jul. 12, 2024

(51) Int. Cl. A61F 5/455; A61M 1/00

(52) U.S. Cl. CPC A61F 5/455; A61M 1/80

(57) ABSTRACT

Example fluid collection devices, systems, and methods of use are described. A fluid collection device includes a handheld body, a fluid permeable member, a conduit, and a pump. The handheld body has a proximal end region at least partially defining an aperture, a distal end region at least partially defining an opening, and an interior region. The fluid permeable member is positioned to extend at least partially across the opening. The conduit is in fluid communication with the fluid permeable member and extends at least partially through the aperture. The pump secured to the handheld body and/or positioned within the interior region of the handheld body. The pump is configured to draw fluid from the fluid permeable member and at least partially through the conduit.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and indwelling urinary catheters, such as a Foley catheter, can be used to address some of these circumstances. However, bed pans and urinary catheters have several problems associated therewith. For example, bed pans can be prone to discomfort, pressure ulcers, spills, and other hygiene issues. Indwelling urinary catheters can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection devices, systems, and methods of use. In an embodiment, a fluid collection device includes a handheld body, a fluid permeable member, a conduit, and a pump. The handheld body has a proximal end region at least partially defining an aperture, a distal end region at least partially defining an opening, and an interior region. The fluid permeable member is positioned to extend at least partially across the opening. The conduit is in fluid communication with the fluid permeable member and extends at least partially through the aperture. The pump is secured to the handheld body and/or positioned within the interior region of the handheld body. The pump is configured to draw fluid from the fluid permeable member and at least partially through the conduit.

In an embodiment, a method of collecting fluid includes holding at least a portion of a proximal end region of a handheld body of a fluid collection device. The handheld body includes a distal end region at least partially defining an opening, an interior region, and an aperture at least partially defined by the proximal end region. The method also includes positioning a fluid permeable member of the fluid collection device at least proximate to a urethral opening of a user while holding at least a portion of the proximal end region of the handheld body of the fluid collection device. The fluid permeable member is positioned to extend at least partially across the opening. The method includes activating a pump secured to the handheld body and/or positioned within the interior region of the handheld body effective to create a negative pressure on an inlet of a conduit in fluid communication with the fluid permeable member. The conduit extends at least partially through the aperture. The method also includes drawing fluid discharged by the urethra through the fluid permeable member, into the inlet of the conduit, and through the conduit extending at least partially through the aperture to remove the fluid from the fluid collection device while the pump is activated to create the negative pressure.

In an embodiment, a fluid collection system includes a fluid collection device, a fluid collection container, and a pump. The fluid collection device includes a handheld body, a fluid permeable member, and a conduit. The handheld body has a proximal end region at least partially defining an aperture, a distal end region at least partially defining an opening, and an interior region. The fluid permeable member is positioned to extend at least partially across the opening. The conduit is in fluid communication with the fluid permeable member and extends at least partially through the aperture. The fluid collection container has an interior region in fluid communication with the conduit. The pump is in fluid communication with the interior region of the fluid collection container and configured to create a negative pressure in the interior region of the fluid collection device effective to draw fluid from the fluid permeable member and at least partially through the conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a system for fluid collection, according to an embodiment.

FIG. 2A is a side view of a fluid collection device, according to an embodiment.

FIG. 2B is a side view of the fluid collection device of FIG. 2A during use, according to an embodiment.

FIG. 3 is an isometric side view of a fluid collection device, according to an embodiment.

FIG. 4A is an isometric side view of a fluid collection device, according to an embodiment.

FIG. 4B is a cross-sectional view of the fluid collection device of FIG. 4A along line 4B.

FIG. 5 is a flow diagram of a method to collect fluid, according to an embodiment.

### DETAILED DESCRIPTION

Embodiments disclosed herein are related to handheld urine collection and pump devices, and related systems and methods. In some embodiments, a handheld fluid (e.g., urine) collection device including a pump is configured to transport urine away from the body as a user discharges the fluid. The fluid collection device may include an all-in-one device having a handheld body, a pump positioned in the handheld body or secured to the handheld body, and an actuator configured to activate and deactivate the pump, according to an embodiment. The pump may be powered by batteries selectively and removably secured to or positioned in the handheld body. The pump may provide suction through a fluid permeable member that is at least one (e.g., all) of ergonomically shaped to interface with female anatomy, disposable, and/or flushable. In some embodiments, the body of the device and the fluid permeable member are configured such that the fluid permeable member may be ejected after use via a button on the body of the device or through a flexible cup shape on the body holding the fluid permeable member. Urine discharged onto and drawn from the fluid permeable member may be directed either into a rigid urine collection container or directly into a toilet.

At least one, some, or all of the fluid collection devices, systems, and methods described herein result in technical effects that improve upon current standards of care. For example, in comparison to conventional diapers and bodily fluid collection pads, many fluid collection devices described herein provide the technical effect of allowing users to avoid the emotional and physical burden that comes from sitting in a wet diaper and/or having the diaper changed through the day. Furthermore, at least one, some, or all of the embodiments of fluid collection devices described herein provide the technical effect of avoiding the risks associated with transferring a patient from a wheelchair to a toilet. Instead, at least one, some, or all of the embodiments described herein provide the technical effect of allowing a patient to remain in a wheelchair, bed, or other stationary position to void urine. Moreover, at least one, some, or all of the embodiments of fluid collection devices described herein provide a more portable, attractive design that can be used only when needed, rather than constantly positioned proximate to the urethral opening in conventional fluid collection devices. At least one, some, or all of the embodiments of fluid collection devices described herein may include easy operation for removal and/or disposal of the fluid permeable member of the device, while retaining the remainder of the device for additional use.

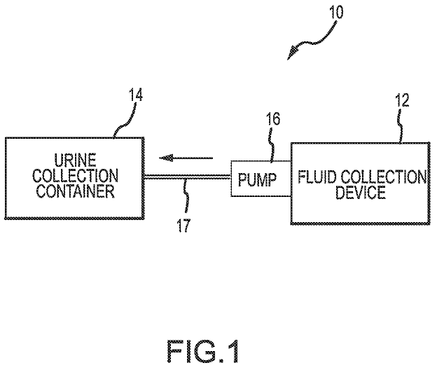

FIG. 1 is a block diagram of a fluid collection system 10, according to an embodiment. The fluid collection system 10 may be included in embodiments of fluid collection systems described herein. The system 10 includes a fluid collection device 12 (e.g., any of the fluid collection devices disclosed herein), a urine collection container 14, and a pump 16 (or vacuum source). The fluid collection device 12, the urine collection container 14, and the pump 16 may be fluidly coupled to each other via one or more conduits 17. In some embodiments, the pump 16 is positioned in an interior region of the body of the fluid collection device 12. In some embodiments, the pump 16 is secured to the body of the fluid collection device 12 outside of the interior region. In some embodiments, the pump 16 may be secured directly to the urine collection container 14. Fluid (e.g., urine or other bodily fluids) collected in the fluid collection device 12 may be removed from the fluid collection device 12 via the conduit 17 secured to the fluid collection device 12. Suction force may be introduced into the chamber of the fluid collection device 12 via the inlet of a conduit (e.g., the conduit 17) between the pump and an opening in the fluid collection device 12 responsive to suction (e.g., vacuum) force applied at the outlet of the conduit 17. The suction force may be applied to the outlet of the conduit 17 by the pump 16 either directly or indirectly. The suction force may be applied directly via the pump 16. For example, the outlet of the conduit 17 may be disposed within the pump 16. An additional conduit 17 may extend from the pump 16 to a point outside of the fluid collection device 12, such as to the urine collection container 14. In such examples, the pump 16 may be disposed between the fluid collection device 12 and the urine collection container 14.

At least a portion of the urine collection container 14 is sized and shaped to retain a fluid therein. The urine collection container 14 may include a bag (e.g., drainage bag), a bottle, a canister, or a cup (e.g., collection jar), or any other enclosed container for storing bodily fluid(s) such as urine. In some examples, the conduit 17 may extend from the fluid collection device 12 or the pump 16 and attach to the urine collection container 14 at a first point therein. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum, suction, or other negative pressure to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

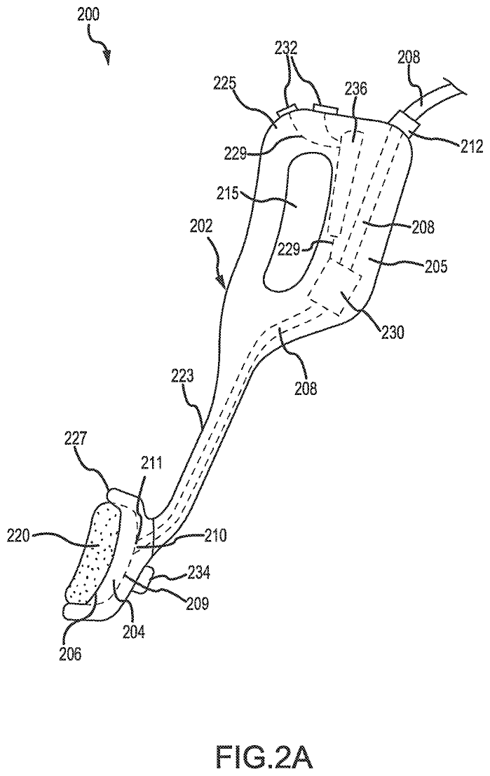

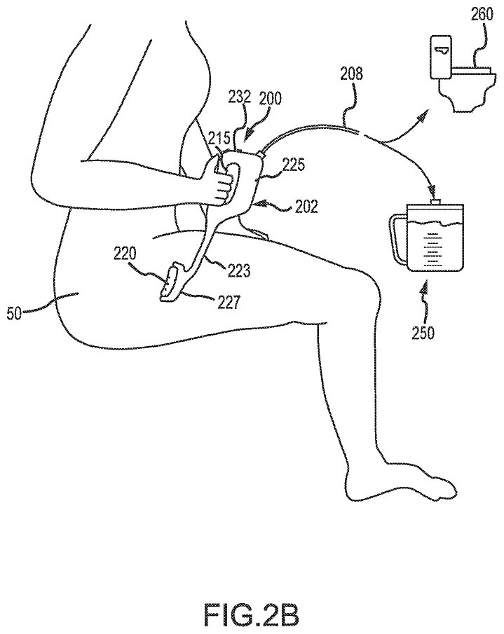

In some embodiments, a fluid collection device may include a urine collection device having a handheld body (e.g. handheld “wand” design) with a configuration that results in the technical effect of providing easy access and placement of a urine collecting region of the urine collection device when a user is in a seated or supine position. Turning to FIG. 2A, which shows a fluid collection device 200 having a handheld body 202, according to an embodiment. The handheld body 202 is ergonomically designed to result in the technical effect of allowing for intuitive single hand operation of the fluid collection device 200. The handheld body 202 may include a proximal end region 225, a distal end region 227 (e.g., urine collecting region), and an interior region 205. The proximal end region 225 may define an aperture 212 or port and the distal end region 227 may at least partially define an opening 206. The handheld body 202 is sized, weighted, and dimensioned to provide the technical effect of allowing a user or caregiver to hold a portion (e.g., the proximal end region 225 with a single hand while the distal end region 227 is positioned at least proximate to the urethra of the user. With the distal end region 227 positioned at least proximate to the urethra of the user, the fluid collection device 200 provides the technical effect of allowing the user or caregiver to hold the proximal end region 225 at a position that is spaced above the legs of the user (e.g., the hand holding the proximal end region 225 is not positioned between the legs of the user when the distal end region 227 is positioned at least proximate to the urethra of the user). For example, the fluid collection device 200 may include a longitudinal length of about 15 cm to about 55 cm, about 15 cm to about 25 cm, about 25 cm to about 35 cm, about 35 cm to about 45 cm, about 45 cm to about 55 cm, about 15 cm to about 20 cm, about 20 cm to about 25 cm, about 25 cm to about 30 cm, about 30 cm to about 35 cm, about 35 cm to about 40 cm, about 40 cm to about 45 cm, about 45 cm to about 50 cm, or about 50 cm to about 55 cm. In some embodiments, the fluid collection device 200 includes a weight of less than about 2 kg, less than about 1.5 kg, less than about 1 kg, or less than about 0.5 kg.

One or more portions of the handheld body 202 may define the interior region 205 of the handheld body 202. In some embodiments, the interior region 205 defined by the handheld body 202 includes one or more regions of void space housing one or more components described below. The proximal end region 225 of the handheld body 202 may at least partially define at least a portion of the interior region 205 and includes a handle 215. The handle 215 is sized and dimensioned to provide the technical effect of allowing a user to grip or hold the handheld body 202 with one hand during use. For example, FIG. 2B, shows a user 50 holding the handheld body 202 during use with a single hand on the handle 215. In some embodiments, the handle 215 includes a through hole for the hand of the user to grip the handheld body 202. The through hole of the handle 215 may include a length of about 5 cm to about 10 cm. In some embodiments, the handle 215 includes other grips or finger holds configured and/or contoured to provide the technical of allowing a user to grip the handheld body 202 with one hand. The handle 215 may include a longitudinal length of about 5 cm to about 15 cm, about 5 cm to about 10 cm, about 10 cm to about 15 cm, about 5 cm to about 7 cm, about 7 cm to about 9 cm, about 9 cm to about 11 cm, about 11 cm to about 13 cm, or about 13 cm to about 15 cm. In some embodiments, at least a portion of the handle 215 may include a cross-sectional width or diameter of 1 cm to about 5 cm, about 1 cm to about 3 cm, about 3 cm to about 5 cm, about 1 cm to about 2 cm, about 2 cm to about 3 cm, about 3 cm to about 4 cm, about 4 cm to about 5 cm, about 2 cm, about 2.5 cm, about 3 cm, about 3.5 cm, about 4 cm, about 4.5 cm, about 5 cm, less than about 5 cm, less than about 4 cm, less than about 3 cm, or less than about 2 cm.

In some embodiments, the distal end region 227 at least partially defines a chamber 204 in fluid communication with the opening 206 defined by the distal end region 227. Components and features of the fluid collection device 200 inside the handheld body 202 are shown in FIG. 2A using broken lines. When the fluid permeable member 220 is removed from the distal end region 227, the chamber 204 may be a region of empty space that is generally void of other material. The chamber 204 may be at least partially enclosed by one or more portions of the distal end region 227. For example, distal end region 227 may be cup-or bowl-shaped with the opening 206 leading into the chamber 204 (e.g., interior region) of the distal end region 227. In these and other embodiments, the distal end region 227 may include a lateral diameter of about 1 cm to about 5 cm, about 1 cm to about 3 cm, about 3 cm to about 5 cm, about 1 cm to about 2 cm, about 2 cm to about 3 cm, about 3 cm to about 4 cm, or about 4 cm to about 5 cm.

In some embodiments, the distal end region 227 is elongated and may be oriented to extend longitudinally in the same direction as the handheld body 202. For example, the distal end region 227 may be substantially cylindrical. In these and other embodiments, the distal end region 227 may have a length about 5 cm to about 18 cm, about 5 cm to about 15 cm, about 5 cm to about 10 cm, about 10 cm to about 15 cm, about 5 cm to about 7.5 cm, about 7.5 cm to about 10 cm, about 10 cm to about 12.5 cm, about 12.5 cm to about 15 cm, or about 15 cm to about 18 cm. The distal end region 227 may include a cross-sectional diameter of about 1 cm to about 5 cm, about 1 cm to about 3 cm, about 3 cm to about 5 cm, about 1 cm to about 2 cm, about 2 cm to about 3 cm, about 3 cm to about 4 cm, or about 4 cm to about 5 cm. In these and other embodiments, the opening 206 may include an elongated opening extending longitudinally with the elongated distal end region 227. The distal end region 227 may include a back surface, a front surface spaced from the back surface and distal to at least a portion of the back surface, and two elongated edges spaced inwardly from the front surface such that the two elongated edges are positioned between the front surface and the back surface. The two elongated edges may at least partially define the longitudinally elongated opening 206 on the front surface. In other embodiments, the distal end region 227 may include other shapes, such as one of more substantially planar surfaces, triangular, or other suitable shape. For example, in some embodiments, the front of the distal end region 227 is generally arched or rounded and the back of the distal end region 227 is generally planar.

The distal end region 227 may be sized and dimensioned to be positioned at least proximate to the opening of the female urethra or over the urethra of a male having a buried penis, and urine may enter the chamber 204 of the distal end region 227 via the opening 206. For example, the opening 206 may exhibit an elongated shape that is configured to extend from a first location below the urethral opening (e.g., at or near the anus or the vaginal opening) to a second location above the urethral opening (e.g., at or near the clitoris or the pubic hair). The opening 206 may exhibit an elongated shape since the space between the legs of a female is relatively small when the legs of the female are closed, thereby only permitting the flow of the fluids along a path that corresponds to the elongated shape of the opening 206. For example, the opening may extend longitudinally along the distal end region 227. The opening 206 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the distal end region 227. The opening 206 may exhibit a width that is greater than 50% of the circumference of the distal end region 227 since the vacuum (e.g., suction) through the conduit 208 pulls the fluid into the conduit 208. In some embodiments, the opening 206 may be vertically oriented (e.g., having a major axis parallel to the longitudinal axis of the distal end region 227). In some embodiments, (not shown), the opening 206 may be horizontally oriented (e.g., having a major axis perpendicular to the longitudinal axis of the distal end region 227). In some embodiments, the inward border or edge of the distal end region 227 defines the opening 206. The edge may include two opposing arced portions, the arc portions following the outer circumference or periphery of the substantially cylindrical distal end region 227.

According to various embodiments, the distal end region 227 may include any of a number of different shapes and configurations, such as the shapes and configurations of the fluid impermeable barriers disclosed in U.S. Patent Application No. 63/171,165 filed on Apr. 6, 2021, U.S. Patent Application No. 63/228,258 filed on Aug. 2, 2021, U.S. Patent Application No. 63/228,252 filed on Aug. 2, 2021, U.S. Patent Application No. 63/228,244 filed on Aug. 2, 2021, U.S. patent application Ser. No. 17/394,055 filed Aug. 4, 2021, or PCT Application No. PCT/US20/40860, the disclosures of each of which are incorporated herein by reference in their entirety.

At least a portion (e.g., all) of the distal end region 227 includes a fluid impermeable barrier. For example, the front surface and the back surface of the distal end region 227 may include the fluid impermeable barrier. In some embodiments, the distal end region 227 includes an internal wall 209 at least partially defining the chamber 204. The internal wall 209 may partially block or prevent fluid communication between the chamber 204 and the interior region 205 of the handheld body 202. In some embodiments, the internal wall 209 defines an aperture 211 or port configured to provide fluid communication between the chamber 204 and a conduit 208 positioned in the interior region 205 of the handheld body 202. In some embodiments the internal wall 209 forms a funnel narrowing to the aperture 211. In many embodiments, the aperture 211 is positioned proximate to the bottom of the chamber 204 that is distal to the proximal end region 225 or handle 215 such that gravity assisting is pulling fluid discharged into the fluid permeable member 220 and the chamber 204 towards the aperture 211 for removal from the chamber 204. Locating the aperture 211 and the inlet 210 at or near a gravimetrically low point of the chamber 204 enables the conduit 208 to receive more of the fluids than if inlet 210 was located elsewhere and reduce the likelihood of pooling (e.g., pooling of the fluids may cause microbe growth and foul odors). For instance, the fluids in the fluid permeable member 220 may flow in any direction due to capillary forces. However, the fluids may exhibit a preference to flow in the direction of gravity, especially when at least a portion of the fluid permeable member 220 is saturated with the fluids.

The fluid impermeable material of the distal end region 227 may allow the distal end region to temporarily store the fluids in the chamber 204. For example, the fluid impermeable barrier of the distal end region 227 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier of the distal end region 227 substantially prevents the fluids from exiting the portions of the chamber 204 that are spaced from the opening 206. The fluid impermeable barrier of the distal end region 227 is flexible, thereby enabling the of the distal end region 227 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier of the distal end region 227 may be air permeable. In such an embodiment, the fluid impermeable barrier of the distal end region 227 may be formed of a hydrophobic material that defines a plurality of pores. In an embodiment, one or more portions of at least the outer surface of the fluid impermeable barrier of the distal end region 227 may be formed from a soft and/or smooth material, thereby reducing chaffing.

In some embodiments, the handheld body 202 includes a neck 223 extending between the distal end region 227 and the proximal end region 225. The neck 223 may be sealed around the conduit 208 or the neck 223 may define at least a portion of the conduit 208. As shown in FIG. 2B, the neck 223 may be elongated such that when the distal end region 227 is positioned at least proximate to the urethra of the user 50, the handle 215 is positioned above the legs of the user (e.g. the handle is not between the legs of the user when the distal end region 227 is positioned at least proximate to the urethra of the user 50). At least one of the neck 223 and the proximal end region 225 may be substantially rigid and/or include a substantially rigid frame or member. In some embodiments, the neck 223 is flexible but retains its shape after flexing.

In some embodiments, the neck 223 includes is flexible and includes a shape memory material the results in the technical effect of allowing the neck 223 to bend to a desired shape or configuration and retain that shape or configuration. The shape memory material may include a shape memory polymer or a metal (e.g., shape memory metal). Suitable shape memory materials are composed to adopt an intermediate or permanent shape in response to a stimuli. The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material, whereinafter the metal wire may be modified to a second shape via physical force applied thereto or via heating.

In an embodiment, the shape memory material may include metal, such as an elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn, or the like; Co—Cr—Ni—Mo alloys (e.g., Elgiloy®) or the like; or any other alloy having shape memory characteristics. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polysulphone, or the like; polynorbonene; other deformable polymers; or any other shape memory polymer.

Returning to FIG. 2A, the fluid collection device 200 also includes a fluid permeable member 220 positioned to extend at least partially across the opening 206, according to an embodiment. For example, the fluid permeable member 220 may be positioned to fill substantially all of the chamber 204 and cover or extend across substantially all of the opening 206. As such, at least a portion the fluid permeable member 220 may be shaped complementary to at least a portion of the chamber 204. In some embodiments, the distal end region 227, the chamber 204, and the fluid permeable member 220 are all complementary elongated. For example, the distal end region 227 and the fluid permeable member 220 may be substantially cylindrical. In these and other embodiments, the fluid permeable body 220 may form an arched surface in the distal end region 227 that is positioned to be placed at least proximate (e.g., adjacent) to the urethra of the user 50.

In use, the fluid permeable member 220 of the fluid collection device is positioned adjacent to a urethra of the user. The fluid permeable member 220 is disposed within a chamber 204 (shown in FIG. 2A) of the distal end region 227 of the fluid collection device 200 and is exposed to the urethra of the user through the opening 206 in the distal end region 227. In some embodiments, the configuration of the fluid permeable member 220 and/or the distal end region 227 results in the technical effect of the fluid permeable member 220 being selectively ejectable from the chamber 204 and the distal end region 227 of the fluid collection device. Accordingly, the fluid permeable member 220 is selectively removable and replaceable at least partially within the chamber 204 in the distal end region 227 of the handheld body 202, according to an embodiment. In some embodiments, the fluid permeable member 220 may be manually pulled from the chamber 204 of the distal end region 227 by gripping the fluid permeable member 220 with a hand or other instrument. In some embodiments, the fluid collection device 200 includes a configuration that results in the technical effect of a user not being required to touch a soiled or used fluid permeable member 220 to remove the fluid permeable member 220 from the chamber of the distal end region 227. This configuration provides a more sanitary process for use and changing of the device. For example, the fluid permeable member 220 may be ejected from the chamber 204 and the distal end region 227 by pushing a back surface of the distal end region 227. In some embodiments, the distal end region 227 may include a flexible material (described above) such as rubber that results in the technical effect of allowing a force exerted on the back surface of the distal end region 227 to push the fluid permeable member 227 out of the chamber 204 and/or turn the distal end region at least partially inside out to eject the fluid permeable body 220 from the distal end region 227. In some embodiments, the fluid collection device 200 includes an actuator 234 (e.g., button) on the distal end region 227 of the handheld body 202. The actuator is configured to at least partially eject the fluid permeable member 220 from the chamber 204 when the actuator 234 is pressed. For example, the actuator 234 may be biased away from the internal wall 209, but when pressed exerts a force on the internal wall 209 and/or extends at least partially into the chamber 204 to push the fluid permeable member 220 out of the chamber 204. When the actuator 234 or released, the actuator may be biased to return to an original position that does not exert a force on the internal wall 209 and/or extend at least partially into the chamber 204.

Once a soiled or used fluid permeable member 220 is ejected or otherwise removed from the chamber 204, a new, replacement fluid permeable member 220 may be inserted into the chamber 204 of the distal end region 227 for use with the original fluid collection device 200. The fluid permeable member 220 may include materials that result in the technical effect of the fluid permeable member 220 being safe to flush down the toilet for disposal of the fluid permeable member 220. The fluid permeable member 220 may be configured to wick any fluid away from the opening 206, thereby preventing the fluid from escaping the chamber 204. The fluid permeable member 220 also may wick the fluid generally towards an interior of the chamber 204 and or the aperture 211. A portion of the fluid permeable member 120 may define a portion of an outer surface of the fluid collection device 200. Specifically, the portion of the fluid permeable member 220 defining the portion of the outer surface of the fluid collection device 200 may be the portion of the fluid permeable member 220 exposed by the opening 206 defined by the distal end region 227.

The fluid permeable member may include one or more fluid permeable materials. For example, the fluid permeable member 220 may include one or more of a polypropylene based material, a polyurethane based foam, a bamboo based material, and/or a nonwoven material. The fluid permeable member 220 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material. The fluid permeable member 220 may include a one-way fluid movement fabric. As such, the fluid permeable member 220 may remove fluid from the area around the female urethra, thereby leaving the urethra dry. The fluid permeable member 220 may enable the fluid to flow generally towards a reservoir (not shown) of void space formed within the chamber 204 and/or towards the inlet 210 of the conduit 208 positioned at the aperture 211 of the internal wall 209. For example, the fluid permeable member 220 may include a porous or fibrous material, such as hydrophilic polyolefin. In some embodiments, the fluid permeable member 220 consists of or consists essentially of a porous or fibrous material, such as hydrophilic polyolefin. Examples of polyolefin that may be used in the fluid permeable member 220 include, but are not limited to, polyethylene, polypropylene, polyisobutylene, ethylene propylene rubber, ethylene propylene diene monomer, or combinations thereof. The porous or fibrous material may be extruded into a substantially cylindrical or other shape to fit within the chamber 204 of the distal end region 227. The fluid permeable member 220 may include varying densities or dimensions. Moreover, the fluid permeable member 220 may be manufactured according to various manufacturing methods, such as molding, extrusion, or sintering.

In some embodiments, the fluid permeable member 220 includes a singular and porous body. In some embodiments, a majority of the outer surface of the fluid permeable member 220 interfaces with the internal wall 209. In some embodiments, the fluid permeable member 220 may include two or more layers of fluid permeable materials and include no (or an absence of) more than two layers of material between the opening 206 and the conduit 208 positioned within the fluid permeable member 220. For example, the fluid permeable member 220 may include a fluid permeable membrane covering or wrapping around at least a portion of a fluid permeable support, with both the fluid permeable membrane and the fluid permeable support being disposed at least partially in the chamber 204. The fluid permeable membrane may cover or extend across at least a portion (e.g., all) of the opening 206. The fluid permeable membrane may be configured to wick any fluid away from the opening 206, thereby preventing the fluid from escaping the chamber 204. The permeable properties referred to herein may be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 206. The material of the fluid permeable membrane and the fluid permeable support also may include natural fibers. In such examples, the material may have a coating to prevent or limit absorption of fluid into the material, such as a water repellent coating. Such “wicking” or other physical properties may not include absorption into the fluid permeable member 220, such as not include absorption of the bodily fluid into the fluid permeable member 220. Put another way, substantially no absorption or solubility of the bodily fluids into the material may take place after the material is exposed to the bodily fluids and removed from the bodily fluids for a time. While no absorption is desired, the term “substantially no absorption” may allow for nominal amounts of absorption of fluid into the wicking material (e.g., absorbency), such as less than about 30 wt % of the dry weight of the fluid permeable member 220, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable member 220. In an embodiment, the fluid permeable member 220 may include at least one absorbent or adsorbent material.

The fluid permeable membrane may also wick the fluid generally towards an interior of the chamber 204. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 200. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable member 220 includes a fluid permeable support including a porous nylon structure (e.g., spun nylon fibers) and a fluid permeable membrane including gauze about or over the porous nylon structure of the fluid permeable support.

Returning to FIG. 2A, the fluid collection device 200 also includes the conduit 208 in fluid communication with the fluid permeable member 220 and extending at least partially through the aperture 212 in the proximal end region 225. In some embodiments, one or more conduit 208 provide fluid communication between the aperture 211 in the distal end region 227 and the aperture 212 in the proximal end region 225. Fluids received in the chamber 204 in the distal end region 227 from the urethra may be removed through the conduit 208. The conduit 208 may extend through the proximal end region 225 and the neck 223. In some embodiments, the conduit 208 includes an inlet 210 that is positioned at least proximate to the aperture 211 in the distal end region 227. For example, the inlet 210 of the conduit 208 may substantially interface a portion of the fluid permeable member 220 positioned in the chamber 204. In some embodiments, the conduit 208 may extend through the aperture 211 and into the fluid chamber 204 of the distal end region such that the inlet 210 is positioned at least partially within the chamber 204. However, in other embodiments, the inlet 210 may be positioned flush with or behind the aperture 211. The fluid collected in the fluid collection device 100 may be removed from the interior region of the chamber 204 via the conduit 208. At least some of the conduit 208 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex. The aperture 211 may be configured to form an at least substantially fluid tight seal against the conduit 208 or the at least one tube thereby substantially preventing the fluids from escaping the chamber 204.

In some embodiments, the fluid permeable member 220 and the chamber 204 may be sized and dimensioned such that a reservoir for temporarily storing fluids is defined in the chamber 204. The reservoir may be an unoccupied portion of the chamber 204 and is void of other material. In some embodiments, the reservoir is defined at least partially by the fluid permeable body 220 and the internal wall 209. For example, in an embodiment, the reservoir may be located at the portion of the chamber 204 that is closest to the inlet 210. However, the reservoir may be located at different locations in the chamber 204. In these and other embodiments, the conduit 208 may extend through the aperture 211 in the internal wall 209 and into the reservoir. In another example, the fluid permeable body 220 is spaced from at least a portion of the conduit 208 and the reservoir may be the space between the fluid permeable body 220 and the conduit 208. In some embodiments, the fluid permeable body 220 fills or occupies substantially all of the chamber 204.

A pump 230 is positioned within the interior region 205 of the handheld body 202 and/or secured to the handheld body 202 of the fluid collection device 200, according to an embodiment. The pump 230 may draw urine voided into the fluid permeable member 220 and the chamber 204 through the conduit 208 and direct the urine to a storage container or a toilet. For example, the pump 230 may create a negative pressure at the inlet 210 effective to draw fluid in the chamber 204 and/or the fluid permeable member 220 into the conduit 208 through the inlet 210. The pump 230 may include at least one of a peristaltic pump, a wetted pump, a positive displacement pump, a manual pump, and electric pump, a diaphragm pump, a centrifugal pump, a displacement pump, a magnetically driven pump, a micro pump, or any pump configured to produce a negative pressure at the inlet 210 of the conduit.

In some embodiments, the pump 230 is positioned in the interior region 205 of the handheld body 202 and may be between two portions of the conduit 208. In some embodiments, some embodiments, the pump 230 is secured to the handheld body 202 on the outside of the handheld body 202, such as on the outside of the proximal end region 225.

The fluid collection device 200 also may include an ergonomically positioned actuator 232 (e.g. one or more buttons) on the handheld body 202 operably coupled to the pump 230 with one or more wires 229 that result in the technical effect of allowing for intuitive single hand operation of the fluid collection device 200. The actuator 232 is configured to selectively activate and deactivate the pump 230. In some embodiments, the actuator 232 is positioned at the proximal end region 225 of the handheld body 202, generally distal to the distal end region 227. In other embodiments, the actuator 232 may be positioned elsewhere on the handle 215 or the neck 223.

In some embodiments, the handheld body 202 houses at least one (e.g., both) of the pump 230 and a power source 236 in the interior region 205 of the handheld body 202. The power source 236 may be secured to and/or positioned within the interior region 205 of the handheld body 202 and operably coupled to the actuator 232 and the pump 230. The power source 236 may include any power source configured to provide power to the pump 230. For example, the power source 236 may include one or more batteries, such as user-rechargeable or replaceable batteries.

Turning to FIG. 2B, which shows the fluid collection device 200 in use with a user 50. Although shown with the user 50 holding the fluid collection device 200 with a single hand, the fluid collection device may similarly be held by a single hand of a caregiver and applied to the user 50. Thus, either the user 50 or a caregiver may hold the fluid collection device 200 to collect fluid from the user 50. The fluid permeable member 220 positioned at least partially within the chamber 204 of the fluid collection device 200 may be positioned at least proximate (e.g., adjacent) to the urethra of the user 50. The user 50 or the caregiver may then activate the pump 230 (not visible) with the actuator 232 and then void urine into the fluid permeable member 220. The pump 230 may pull the voided urine in the fluid permeable member 220 and the chamber 204 into the conduit 208 (not visible). The pump 230 may further direct the urine in the conduit 208 out of the handheld body 202 to at least one of a toilet 260 or a fluid collection container 250. In some embodiments, the conduit 208 is detachable secured to the fluid collection container 250. After the user has finished voiding urine, the distal end region 227 may be moved away from the urethra of the user 50, and the soiled fluid permeable member 220 may be ejected or otherwise removed from the distal end region 227 of the handheld body 202 by the user 50 or the caregiver and disposed. A fresh fluid permeable member 220 may then be positioned at least partially in the chamber 204 of the distal end region 227 to replace the soiled fluid permeable member 220 that has been removed and disposed.

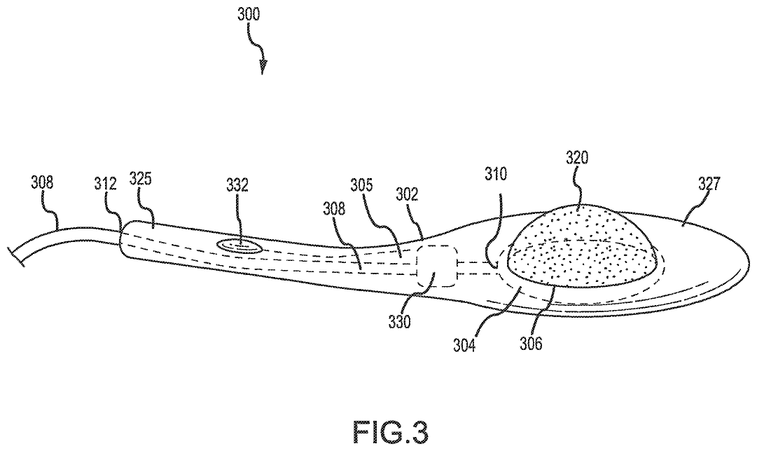

Turning ahead in the drawings to FIG. 3, in some embodiments, a fluid collection device 300 may include a handheld body 302 and a fluid permeable member 320 that is a compressible material that expands after compression, thereby providing the technical effect of allowing the fluid permeable member 320 to be positioned at least proximate to the urethra of a user. Unless otherwise noted, the fluid collection device 300 may include any aspect, materials, features, dimensions, etc., of the fluid collection device 200. For example, the handheld body 302 may include a proximal end region 325 at least partially defining an aperture 312, a distal end region 327 at least partially defining an opening 306, and an interior region 305. The fluid permeable member 320 may be positioned to extend at least partially across the opening 306, and a conduit 308 in fluid communication with the fluid permeable member 320 extends at least partially through the aperture 312 at the proximal end region 327. A pump 330 may be secured to the handheld body 302 and/or positioned within the interior region 305 of the handheld body 302. The pump 330 may be configured to draw fluid from the fluid permeable member 320 and at least partially through the conduit 308. Though not shown in FIG. 3, the fluid collection device 300 may include a power source, similar or the same as the power source 236 in the fluid collection device 200.

In some embodiments, the handheld body 302 may include a substantially spoon-shaped front and rear profile. For example, the proximal end region 325 of the handheld body 302 may include a generally elongated tubular proximal end region and the distal end region 327 includes a generally oval profile. The opening 306 in distal end region 327 may include a generally oval opening positioned within the generally oval profile of the distal end region 327, according to an embodiment. The distal end region 327 may at least partially define a chamber 306 recessed into the distal end region 327. The handheld body 302 may include be substantially flat on the front and the rear of the handheld body 302, effective to provide the technical effect that results in allowing the fluid collection device 300 to be slid more easily into place with the fluid permeable member 320 at least proximate to the urethra.

The fluid permeable member 320 may include any aspect of the fluid permeable member 220. In some embodiments, the fluid permeable member 320 protrudes from the front surface of the distal end region 327 and includes a resilient and compressible fluid permeable foam sized and dimensioned to fit between labia of a user and over or at least proximate to a urethral opening of the user. In some embodiments, the fluid permeable member 320 is substantially flat when sliding the fluid collection device 300 into place with the fluid permeable member 320 at least proximate to the urethra of the user. For example, the fluid permeable member 320 may be substantially flat until a cover (not shown) is removed from the fluid permeable member 320 and the fluid permeable member 320 expands to protrude from the front surface of the distal end region 327. It some embodiments, the fluid permeable member 320 compresses to be substantially flat as the fluid collection device 300 is slid into place, and then the fluid permeable member 320 expands to fit between labia of a user and over or at least proximate to a urethral opening of the user. In some embodiments, the fluid permeable member 320 may inflated to expand and protrude from the distal end region 327 when the fluid permeable member 320 is positioned at least proximate to the urethra of the user. For example, the pump 330 may include a selectively reversible pump that selectively exerts a positive pressure from the inlet 310 to inflate the fluid permeable member 320 to protrude from the distal end region 327. In some embodiments, the fluid permeable member 320 includes a peripheral lip positioned within the chamber 306 that engages with front of the distal end region 327 to inhibit the fluid permeable member 320 from falling out and separating from the handheld body 302 without additional force.

In some embodiments, the fluid permeable member 320 is selectively removable or ejectable, as described above in relation to the fluid permeable member 220 in the fluid collection device 200. Although not visible in FIG. 3, the fluid collection device 300 may include an actuator on the distal end region 327 of the handheld body 302 that is configured to at least partially eject the fluid permeable member 320 from the chamber 306 when the actuator is pressed.

The fluid collection device 300 further includes the conduit 308 having an inlet 310 in fluid communication with the chamber 304 at the distal end region 327, according to an embodiment. The inlet 310 may be positioned at least proximate to and/or in the chamber 304. In some embodiments, the inlet 310 interfaces a portion of the fluid permeable member 320 positioned in the chamber 304 and/or is flush with portion of the distal end region 327 defining the chamber 304. The conduit 308 may include one or more members extending through the interior region 305 of the fluid collection device 300 before extending through the aperture 312 at the proximal end region 325.

The fluid collection device 300 further includes the pump 330 positioned in the interior region 305 of the handheld body 302 and/or secured to the handheld body 302, according to various embodiments. The pump 330 may include any aspect of the pump 230, and may be configured to draw fluid from the fluid permeable member 320 and the chamber 306 and direct or advance fluid out of the fluid collection device 300 through the conduit 308. The fluid collection device 332 may further include an actuator 332 positioned on the handheld body 302 and operably coupled to the pump 330. The actuator 332 is configured to selectively activate and deactivate the pump 330, according to an embodiment. The fluid collection device 300 may be used similar to the fluid collection device 200, described above. In some embodiments, the pump 330 is positioned outside handheld body 302. For example, the pump 330 may be secured to the outside of the handheld body 302. In some embodiments, the pump 330 includes an exterior pump, such as a wall mounted pump or a pump in a housing that is separate from the fluid collection device 300. For example, the pump 330 may be in fluid communication with a fluid collection container which is fluid communication with the fluid collection device 300. When the pump 330, the pump 330 may create a negative pressure in the fluid collection container, which creates a negative pressure at the inlet 310 effective to pull urine in the chamber 304 and the fluid permeable member 320 into the conduit 308 and the fluid collection container.

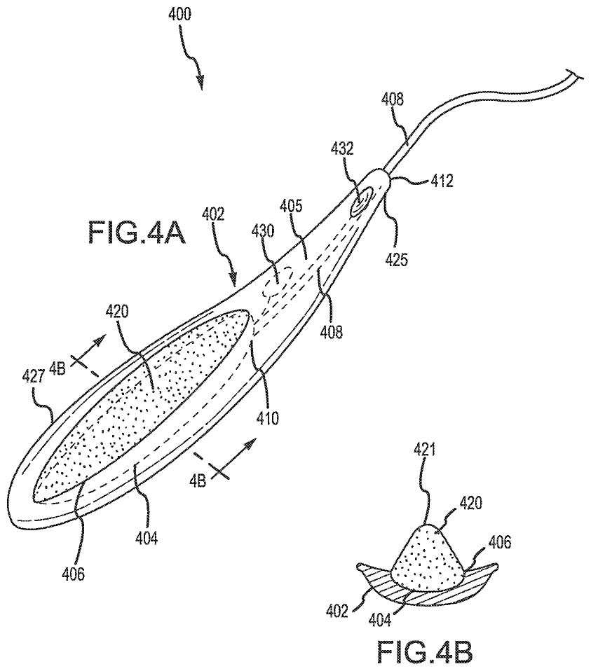

Turning ahead in the drawings to FIG. 4A, in some embodiments, a fluid collection device 400 may include a handheld body 402 and a fluid permeable member 420 that tapers at two opposing ends and towards a top of the fluid permeable member 420, resulting in the technical effect of the fluid permeable member 420 fitting comfortably between labia of the user. Unless otherwise noted, the fluid collection device 400 may include any aspect, materials, features, dimensions, etc., of the fluid collection devices 200, 300. For example, the handheld body 402 may include a proximal end region 425 at least partially defining an aperture 412, a distal end region 427 at least partially defining an opening 406, and an interior region 405. The fluid permeable member 420 may be positioned to extend at least partially across the opening 406, and a conduit 408 in fluid communication with the fluid permeable member 420 extends at least partially through the aperture 412 at the proximal end region 427. A pump 430 may be secured to the handheld body 402 and/or positioned within the interior region 405 of the handheld body 402. The pump 430 may be configured to draw fluid from the fluid permeable member 420 and at least partially through the conduit 408. Though not shown in FIG. 4A, the fluid collection device 400 may include a power source, similar or the same as the power source 236 in the fluid collection device 200.

In some embodiments, the handheld body 402 may be substantially spoon-, pear-, or tear drop-shaped or pear-shaped. For example, the proximal end region 425 of the handheld body 402 may include a generally elongated tubular proximal end region and the distal end region 427 includes a generally elongated oval profile. The opening 406 in distal end region 427 may include a generally elongated opening positioned within the generally elongated oval profile of the distal end region 427, according to an embodiment. The distal end region 427 may at least partially define a chamber 406 recessed into the distal end region 427.

The fluid permeable member 420 may include any aspect of the fluid permeable member 220. Turning to the cross-sectional view of FIG. 4B, in some embodiments, the fluid permeable member 420 protrudes from the front surface of the distal end region 427 and may include a resilient and compressible fluid permeable foam sized and dimensioned to fit between labia of a user and over or at least proximate to a urethral opening of the user. The fluid permeable member 420 may be positioned partially within the chamber, and include an elongated ridge 421 at the top of the fluid permeable member 420 and spaced from the distal end region 427. The fluid permeable member 420 may be elongated and taper to two opposing end regions. In some embodiments, the fluid permeable member 420 includes a substantially rounded corner triangular cross-sectional profile, and the distal end region 427 may partially overlap with bottom edges of the fluid permeable member 420 to at least selectively retain the fluid permeable member partially in the chamber 406. In some embodiments, the fluid permeable member 420 is selectively removable or ejectable, as described above in relation to the fluid permeable member 220 in the fluid collection device 200. Although not visible in FIGS. 4A and 4B, the fluid collection device 400 may include an actuator on the distal end region 427 of the handheld body 402 that is configured to at least partially eject the fluid permeable member 420 from the chamber 406 when the actuator is pressed.

The fluid collection device 400 further includes the conduit 408 having an inlet 410 in fluid communication with the chamber 404 at the distal end region 427, according to an embodiment. The inlet 410 may be positioned at least proximate to and/or in the chamber 404. In some embodiments, the inlet 410 interfaces a portion of the fluid permeable member 420 positioned in the chamber 404 and/or is flush with portion of the distal end region 427 defining the chamber 404. The conduit 408 may include one or more members extending through the interior region 405 of the fluid collection device 400 before extending through the aperture 412 at the proximal end region 425.

The fluid collection device 400 further includes the pump 430 positioned in the interior region 405 of the handheld body 402 and/or secured to the handheld body 402, according to various embodiments. The pump 430 may include any aspect of the pump 230, and may be configured to draw fluid from the fluid permeable member 420 and the chamber 406 and direct or advance fluid out of the fluid collection device 400 through the conduit 408. The fluid collection device 400 may further include an actuator 432 positioned on the handheld body 402 and operably coupled to the pump 430. The actuator 432 is configured to selectively activate and deactivate the pump 430, according to an embodiment. The fluid collection device 400 may be used similar to the fluid collection device 200, described above.

In some embodiments, the pump 430 is positioned outside handheld body 402. For example, the pump 430 may be secured to the outside of the handheld body 402. In some embodiments, the pump 430 includes an exterior pump, such as a wall mounted pump or a pump in a housing that is separate from the fluid collection device 400. For example, the pump 430 may be in fluid communication with a fluid collection container which is fluid communication with the fluid collection device 400. When the pump 430, the pump 430 may create a negative pressure in the fluid collection container, which creates a negative pressure at the inlet 410 effective to pull urine in the chamber 404 and the fluid permeable member 420 into the conduit 408 and the fluid collection container.

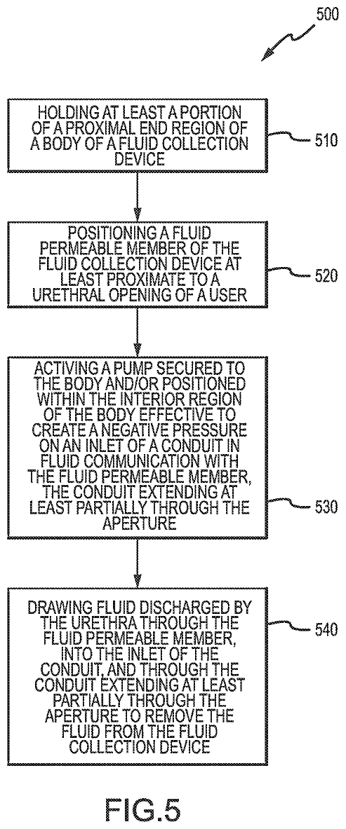

FIG. 5 is a flow diagram of a method 500 to collect fluid, according to an embodiment. The method 500 can include use of any of the fluid collection devices described herein. The method 500 can include act 510 of holding at least a portion of a proximal end region of a handheld body of a fluid collection device. The handheld body includes a distal end region at least partially defining an opening, an interior region, and an aperture at least partially defined by the proximal end region. The method 500 can include an act 520 of positioning a fluid permeable member of the fluid collection device at least proximate to a urethral opening of a user while holding at least a portion of the proximal end region of the body of the fluid collection device. The fluid permeable member may be positioned to extend at least partially across the opening. The method 500 can include an act 530 of activating a pump secured to the body and/or positioned within the interior region of the body effective to create a negative pressure on an inlet of a conduit in fluid communication with the fluid permeable member. The conduit may extend at least partially through the aperture. The method 500 can include an act 540 of drawing fluid discharged by the urethra through the fluid permeable member, into the inlet of the conduit, and through the conduit extending at least partially through the aperture to remove the fluid from the fluid collection device while the pump is activated to create the negative pressure.

In some embodiments, the act 530 includes activating the pump secured to the body and/or positioned within the interior region of the body with an actuator positioned on the body and operably coupled to the pump. In these and other embodiments, the act 510 may include holding a handle at the proximal end region of the handheld body, the distal end region at least partially defining a chamber, the handheld body including a neck extending between the distal end region and the proximal end region, and the conduit extending through the proximal end region and the neck and including the inlet positioning proximate to the chamber and in fluid communication with the chamber. In these and other embodiments, the method 500 may further include an act of inserting the fluid permeable member at least partially into the chamber before positioning the fluid permeable member of the fluid collection device at least proximate to the urethral opening of the user. In some embodiments, the method 500 may further include acts of removing the fluid permeable member from the chamber after at least some of the fluid has been removed from the fluid collection device and positioning a replacement fluid permeable member at least partially into the chamber. In some embodiments, the act of removing the fluid permeable member from the chamber includes pressing an actuator positioned on the distal end region and configured to at least partially eject the fluid permeable member from the chamber. In some embodiments, the act 520 includes positioning the fluid permeable member protruding from a front surface of the distal end region between labia of the user and at least proximate to the urethral opening of the user.

The acts of the method 500 are for illustrative purposes. For example, the acts of the method 500 can be performed in different orders, split into multiple acts, modified, supplemented, or combined. In an example, one or more of the acts of the method 500 can be omitted from the method 500. Any of the acts of the method 500 can include using any of the fluid collection devices and systems, or components of the same disclosed herein.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A fluid collection device, comprising:
a handheld body having a proximal end region at least partially defining an aperture, a distal end region at least partially defining an opening and a chamber, and an interior region;
a fluid permeable member positioned to extend at least partially across the opening, wherein the fluid permeable member is selectively removable and replaceable at least partially within the chamber in the distal end region of the handheld body;
a first actuator on the distal end region of the handheld body and configured to at least partially eject the fluid permeable member from the chamber when the first actuator is pressed;
a conduit in fluid communication with the fluid permeable member and extending at least partially through the aperture; and
a pump secured to the handheld body and/or positioned within the interior region of the handheld body, the pump configured to draw fluid from the fluid permeable member and at least partially through the conduit.

2. The fluid collection device of claim 1, further comprising a second actuator positioned on the handheld body and operably coupled to the pump, the second actuator configured to selectively activate and deactivate the pump.

3. The fluid collection device of claim 2, wherein the proximal end region of the handheld body defines at least a portion of the interior region and includes a handle, the handheld body includes a neck extending between the distal end region and the proximal end region, and the conduit extends through the proximal end region and the neck and includes an inlet proximate to the chamber and in fluid communication with the chamber.

4. The fluid collection device of claim 3, wherein the pump is positioned in the interior region of the handle.

5. The fluid collection device of claim 3, wherein the pump is secured to the proximal end region of the handheld body.

6. (canceled)

7. The fluid collection device of claim 1, wherein the distal end region is generally cup-shaped and shaped complementary to a portion of the fluid permeable member positioned in the chamber.

8. The fluid collection device of claim 1, wherein the fluid permeable member is generally cylindrical and the distal end region is generally cylindrical.

9. The fluid collection device of claim 7, wherein the inlet of the conduit substantially interfaces a portion of the fluid permeable member positioned in the chamber.

10. (canceled)

11. The fluid collection device of claim 3, wherein the proximal end region includes a generally rigid material and the distal end region of the handheld body includes a pliable rubber or silicone material.

12. (canceled)

13. (canceled)

14. (canceled)

15. The fluid collection device of claim 1, wherein the proximal end region includes a generally elongated tubular proximal end region and the distal end region includes a generally oval profile and the opening includes a generally oval opening positioned within the generally oval profile.

16. The fluid collection device of claim 15, wherein the fluid permeable member protrudes from the front surface of the distal end region and includes a resilient and compressible fluid permeable foam sized and dimensioned to fit between labia of a user and at least proximate to a urethral opening of the user.

17. The fluid collection device of claim 1, wherein the handheld body is elongated, the opening is elongated, and the fluid permeable member is elongated and protrudes from distal end region of the handheld body, the fluid permeable member include an elongated ridge spaced from the distal end region and two opposing tapered end regions.

18. (canceled)

19. (canceled)

20. (canceled)

21. The fluid collection device of claim 15, wherein the conduit extends through the proximal end region and includes an inlet proximate to the chamber and in fluid communication with the chamber.

22. (canceled)

23. (canceled)

24. (canceled)

25. The fluid collection device of claim 1, further comprising a fluid collection container in fluid communication with the conduit and positioned to collect fluid pump from the fluid permeable member and through the conduit.

26. A method of collecting fluid, the method comprising:
holding at least a portion of a proximal end region of a handheld body of a fluid collection device, the handheld body including a distal end region at least partially defining an opening and a chamber, an interior region, and an aperture at least partially defined by the proximal end region;
positioning a fluid permeable member of the fluid collection device at least proximate to a urethral opening of a user while holding at least a portion of the proximal end region of the handheld body of the fluid collection device, the fluid permeable member positioned to extend at least partially across the opening;
activating a pump secured to the handheld body and/or positioned within the interior region of the handheld body effective to create a negative pressure on an inlet of a conduit in fluid communication with the fluid permeable member, the conduit extending at least partially through the aperture;
drawing fluid discharged by the urethra through the fluid permeable member, into the inlet of the conduit, and through the conduit extending at least partially through the aperture to remove the fluid from the fluid collection device while the pump is activated to create the negative pressure;
removing the fluid permeable member from the chamber after at least some of the fluid has been removed from the fluid collection device, wherein removing the fluid permeable member from the chamber includes pressing a first actuator positioned on the distal end region and configured to at least partially eject the fluid permeable member from the chamber; and
positioning a replacement fluid permeable member at least partially into the chamber.

27. The method of claim 26, wherein activating a pump secured to the handheld body and/or positioned within the interior region of the handheld body includes activating the pump secured to the handheld body and/or positioned within the interior region of the handheld body with a second actuator positioned on the handheld body and operably coupled to the pump.

28. The method of claim 27, wherein holding at least a portion of a proximal end region of a handheld body of a fluid collection device includes holding a handle at the proximal end region of the handheld body, the handheld body including a neck extending between the distal end region and the proximal end region, and the conduit extending through the proximal end region and the neck and including the inlet positioning proximate to the chamber and in fluid communication with the chamber.

29. The method of claim 28, further comprising inserting the fluid permeable member at least partially into the chamber before positioning the fluid permeable member of the fluid collection device at least proximate to the urethral opening of the user.

30. (canceled)

31. (canceled)

32. The method of claim 26, wherein positioning a fluid permeable member of the fluid collection device at least proximate to a urethral opening of a user includes positioning the fluid permeable member protruding from a front surface of the distal end region between labia of the user and at least proximate to the urethral opening of the user.

33. A fluid collection system, comprising:
a fluid collection device including:
a handheld body having a proximal end region at least partially defining an aperture, a distal end region at least partially defining an opening and a chamber, and an interior region;
a fluid permeable member positioned to extend at least partially across the opening, wherein the fluid permeable member is selectively removable and replaceable at least partially within the interior region of the handheld body;
a first actuator on the distal end region of the handheld body and configured to at least partially eject the fluid permeable member from the chamber when the first actuator is pressed; and
a conduit in fluid communication with the fluid permeable member and extending at least partially through the aperture;
a fluid collection container having an interior region in fluid communication with the conduit; and
a pump in fluid communication with the interior region of the fluid collection container and configured to create a negative pressure in the interior region of the fluid collection device effective to draw fluid from the fluid permeable member and at least partially through the conduit.

34. The fluid collection system of claim 33, wherein the proximal end region of the handheld body defines at least a portion of the interior region and includes a handle, the handheld body includes a neck extending between the distal end region and the proximal end region, and the conduit extends through the proximal end region and the neck and includes an inlet proximate to the chamber and in fluid communication with the chamber.

35. (canceled)

36. The fluid collection system of claim 33, wherein the distal end region is generally cup-shaped and shaped complementary to a portion of the fluid permeable member positioned in the chamber.

37. The fluid collection system of claim 33, wherein the fluid permeable member is generally cylindrical and the distal end region is generally cylindrical.

38. (canceled)

39. (canceled)

40. The fluid collection system of claim 34, wherein the proximal end region includes a generally rigid material and the distal end region of the handheld body includes a pliable rubber or silicone material.

41. (canceled)

42. (canceled)

43. The fluid collection system of claim 33, wherein the proximal end region includes a generally elongated tubular proximal end region and the distal end region includes a generally oval profile and the opening includes a generally oval opening positioned within the generally oval profile.

44. The fluid collection system of claim 43, wherein the fluid permeable member protrudes from the front surface of the distal end region and includes a resilient and compressible fluid permeable foam sized and dimensioned to fit between labia of a user and at least proximate to a urethral opening of the user.

45. The fluid collection system of claim 33, wherein the handheld body is elongated, the opening is elongated, and the fluid permeable member is elongated and protrudes from distal end region of the handheld body, the fluid permeable member include an elongated ridge spaced from the distal end region and two opposing tapered end regions.

46. The fluid collection system of claim 43, wherein the conduit extends through the proximal end region and includes an inlet proximate to the chamber and in fluid communication with the chamber.

47. (canceled)

48. (canceled)

49. (canceled)

50. (canceled)
